(19) United States
(12) Patent Application Publication
(10) Pub. No.: US 2026/0060721 A1
(43) Pub. Date: Mar. 5, 2026

(54) BONE REPOSITIONING SYSTEM AND RELATED METHODS

(71) Applicant: DePuy Synthes Products, Inc., Raynham, MA (US)

(72) Inventors: Todd J. Kent, Cherry Hill, NJ (US); Michael Wahl, Wilmington, DE (US); Richard W. Castle, Honey Brook, PA (US); Kevin Clancy, West Chester, PA (US); Scott Lavoritano, West Chester, PA (US)

(21) Appl. No.: 19/312,722

(22) Filed: Aug. 28, 2025

Related U.S. Application Data
(60) Provisional application No. 63/690,908, filed on Sep. 5, 2024.

(51) Int. Cl. A61B 17/66; A61B 17/00; A61B 17/56; A61B 17/64

(52) U.S. Cl. CPC A61B 17/66; A61B 17/6425; A61B 17/6475; A61B 2017/00477; A61B 2017/565

(57) ABSTRACT

A bone correction system is configured to attach to the medial cuneiform and the first metatarsal of a patient's foot in a deformed configuration. An articulation joint is configured to rotate and angulate the first metatarsal with respect to the medial cuneiform. A translation assembly is configured to cause the first metatarsal to travel toward and/or against the medial cuneiform. Thus, the bone correction system is configured to iterate the patient's foot from the deformed configuration to a corrected configuration. A bone plate can permanently fix the medial cuneiform and the first metatarsal in the corrected configuration.

### CROSS-REFERENCE TO RELATED APPLICATIONS

This claims priority to U.S. Patent Application Ser. No. 63/690,908 filed Sep. 5, 2024, the disclosure of which is hereby incorporated by reference as if set forth in its entirety herein.

### TECHNICAL FIELD

The present invention generally relates to surgical apparatus and methods for correcting alignment between two bones and a joint and particularly relates to surgical systems and procedures for correcting a bunion in a patient's foot.

### BACKGROUND

Bone misalignment and/or deformation can be a source of discomfort and reduced mobility in patients, particularly in a patient's feet. One particularly common foot disorder is a bunion, also known as Hallux valgus. Bunions are a progressive disorder, typically beginning with a leaning of the great toe. The leaning of the great toe may gradually change an angle of the bones and produce a characteristic bump on the medial side of the metatarsal near the joint of the metatarsal with the proximal phalanx. Specifically, the bunion is the prominence made of bone and at times an inflamed bursa. Hallux valgus is the condition in which the great toe deviates from the normal position toward the direction of the second toe. Bunion correction can include rotation of the metatarsal relative to the cuneiform, and can also include translation of the first metatarsal toward the second metatarsal to reduce the intermetatarsal (IM) angle, or angle defined by the longitudinal axes of the of the first and second metatarsals as shown on an anterior-posterior (AP) image.

Bunion correction or repair is a common surgery with over 100,000 surgeries performed annually in the US. Many surgical procedures for bunion repair are invasive and painful, requiring an incision of several inches and a long period of convalescence, of up to 10-12 weeks. Often, an osteotomy is performed at the tarsometatarsal (TMT) joint. In particular, the distal cuneiform and proximal metatarsal can be resected, thereby allowing for corrective movement of the metatarsal with respect to the cuneiform.

Minimally invasive surgery has been performed in orthopedics for decades. One common procedure is known as a Lapidus bunionectomy. In a Lapidus bunionectomy, the bunion is corrected at the great toe by adjusting alignment at the first tarsometatarsal joint. The metatarsal can also be stabilized using bone screws and/or a plate to facilitate fusion between the metatarsal and the medial cuneiform.

In one conventional approach, K-wires are be driven into the metatarsal and the cuneiform, and the metatarsal is rotated with respect to the cuneiform to a corrected rotational position by manipulating the K-wires, akin to a joystick. In order to reduce the IM angle to achieve a corrected IM angle, a clamp is conventionally positioned so that a first arm bears against the medial aspect of the first metatarsal, and a second arm bears against the lateral aspect of the second metatarsal. Bringing the first and second arms closer together thereby reduces the IM angle to a corrected angular position. A bone plate can be affixed to the metatarsal and the cuneiform, thereby fixing the corrected rotational and angular positions of the metatarsal.

While the above-described methods and systems can be suitable for bunion correction, what is desired is a simplified method and apparatus for bunion correction surgery.

### SUMMARY

The foregoing summary is illustrative only and is not intended to be limiting. Other aspects, features, and advantages of the systems, devices, and methods and/or other subject matter described in this application will become apparent in the teachings set forth below. The summary is provided to introduce a selection of some of the concepts of this disclosure. The summary is not intended to identify key or essential features of any subject matter described herein.

According to one example, a bone correction system can include a first bone attachment member having at least one first temporary bone fixation aperture configured to receive a respective at least one first temporary bone fixation member to attach the first bone attachment member to a medial cuneiform. The bone correction system can further include a second bone attachment member having at least one second temporary bone fixation aperture configured to receive a respective at least one second temporary bone fixation member to attach the second bone attachment member to a first metatarsal The bone correction system can further include a joint connected between the first bone attachment portion and the second bone attachment portion, wherein the joint is configured to articulate the second bone attachment member relative to the first bone attachment member in at least two orthogonal planes.

### BRIEF DESCRIPTION OF THE DRAWINGS

Various examples are depicted in the accompanying drawings for illustrative purposes, and should in no way be interpreted as limiting the scope of the examples. Various features of different disclosed examples can be combined to form additional examples, which are part of this disclosure.

FIG. 1A is a perspective view of a patient's foot in a deformed configuration;

FIG. 1B is a perspective view of the patient's foot of FIG. 1A, but shown in a corrected configuration;

FIG. 2A is a front view of the patient's foot of FIG. 1A, showing intermetatarsal angle adjustment;

FIG. 2B is a top view of the patient's foot of FIG. 1A, showing an intermetatarsal angle;

FIG. 3A is a perspective view of a bone correction system constructed in accordance with one example;

FIG. 3B is another perspective view of the bone correction system of FIG. 3A;

FIG. 4A is an exploded perspective view of an articulation assembly of the bone correction system of FIG. 3A including an articulation joint;

FIG. 4B is a sectional side elevation view of the articulation assembly of FIG. 4A;

FIG. 5A is an exploded perspective view of a translation assembly of the bone correction system of FIG. 3A;

FIG. 5B is another exploded perspective view of a translation assembly of the bone correction system of FIG. 5A;

FIG. 5C is a cross-sectional view of the translation assembly of FIG. 5A;

FIG. 5D is a perspective view of the bone correction system including the translation assembly of FIG. 5A;

FIG. 6A is a perspective view of the patient's foot in the deformed configuration and the bone correction system aligned for attachment to the patient's foot;

FIG. 6B is a perspective view similar to FIG. 6A, but showing temporary fixation members aligned to be driven through the bone correction system and into the patient's foot;

FIG. 6C is a perspective view similar to FIG. 6B, but showing the temporary fixation members driven through the bone correction system and into the patient's foot;

FIG. 6D is a perspective view similar to FIG. 6C, but showing the bone correction system in a configuration that has moved the patient's foot to the corrected configuration shown in FIG. 1B; and

FIG. 7 is a perspective view similar to FIG. 6D, but showing the bone correction system and temporary fixation members removed, and showing a bone plate that fixes the medial cuneiform to the first metatarsal.

### DETAILED DESCRIPTION

The various features and advantages of the systems, devices, and methods for bone repositioning described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

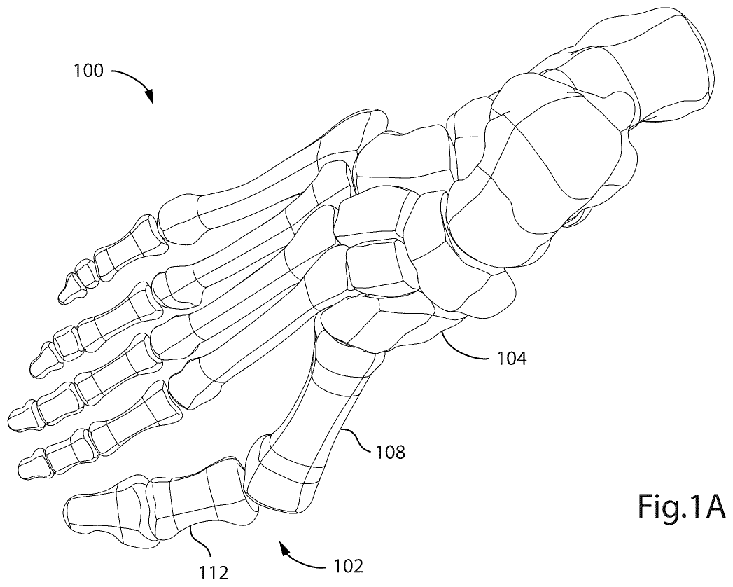

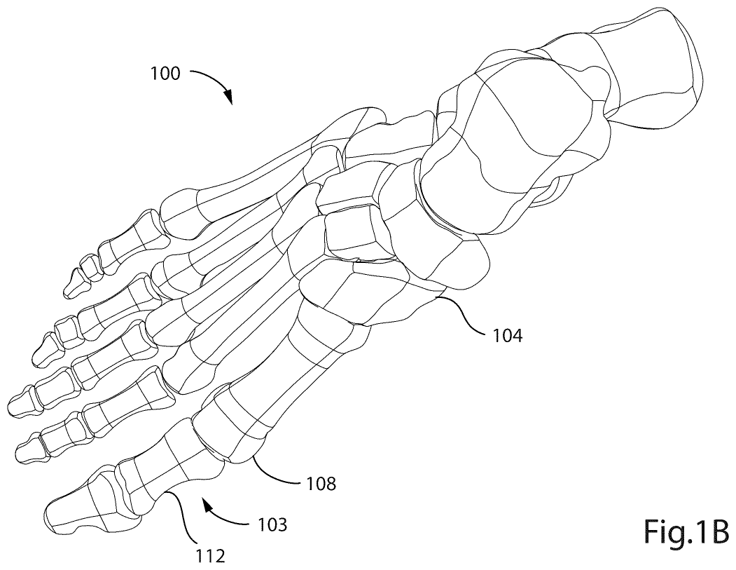

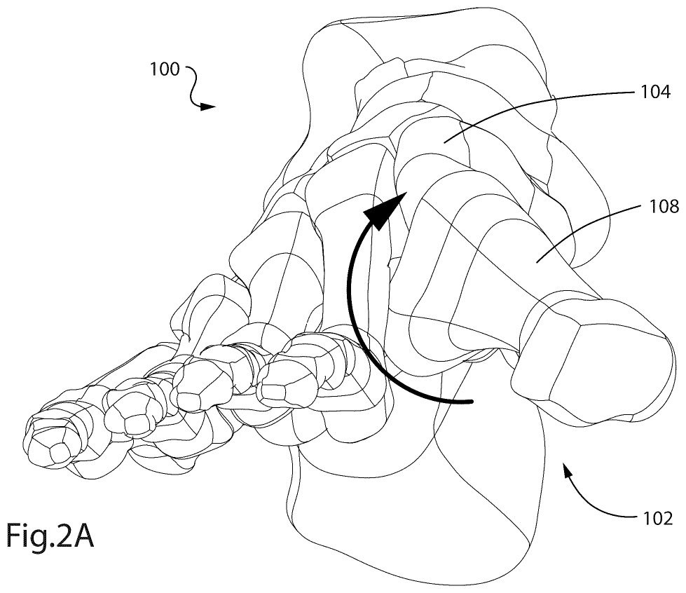

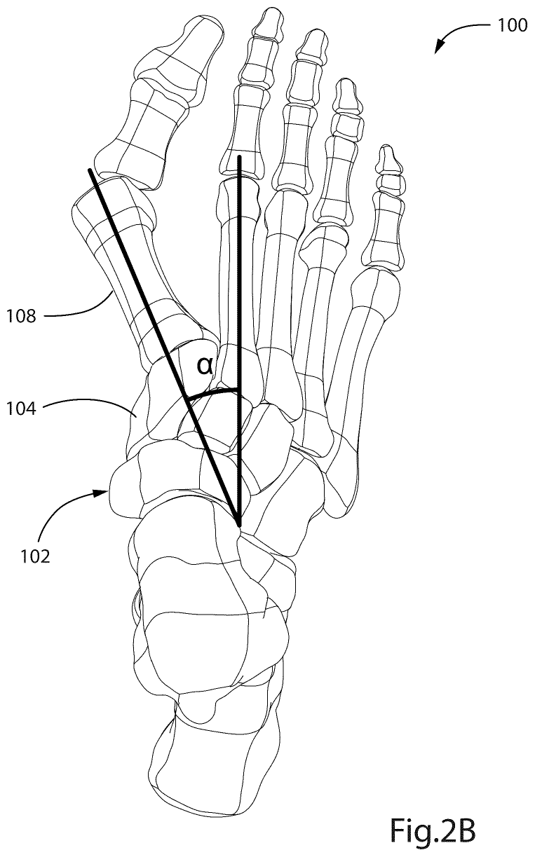

FIG. 1A shows a skeletal view of a patient's foot 100 having one or more bones in a deformed configuration 102. The deformed configuration 102 can be a bunion, as illustrated. The deformed configuration 102 can be a misalignment between a metatarsal 108 and a proximal phalanx 112 of the patient's great toe. In particular, as shown at FIG. 2A, the metatarsal can be rotationally misaligned, such that counter-rotation of the metatarsal 108 can move the patient's foot to a rotationally corrected configuration. As shown at FIG. 2B the IM angle α is defined by the first metatarsal 108 and a second metatarsal adjacent the first metatarsal 108. When the patient's foot 100 is in the deformed configuration, the IM angle can be greater than a typical or “normal” IM angle, which is generally less than 11 degrees, such as less than 9 degrees. Generally, an IM angle greater than nine degrees can be considered to be moderately deformed, and an IM angle greater than 11 degrees can be considered to be severely deformed. The IM angle shown at FIG. 2B is fifteen degrees. The rotational and angularly deformed configuration of the patient's foot can cause the metatarsal 108 to be at an angle with respect to the proximal phalanx 112. A high degree of misalignment between the metatarsal 108 and the proximal phalanx 112 can lead to severe pain and rubbing and discomfort and other problems in the patient's foot 100. Accordingly, it can be beneficial to correct the alignment between the metatarsal 108 and the proximal phalanx 112 of the great toc.

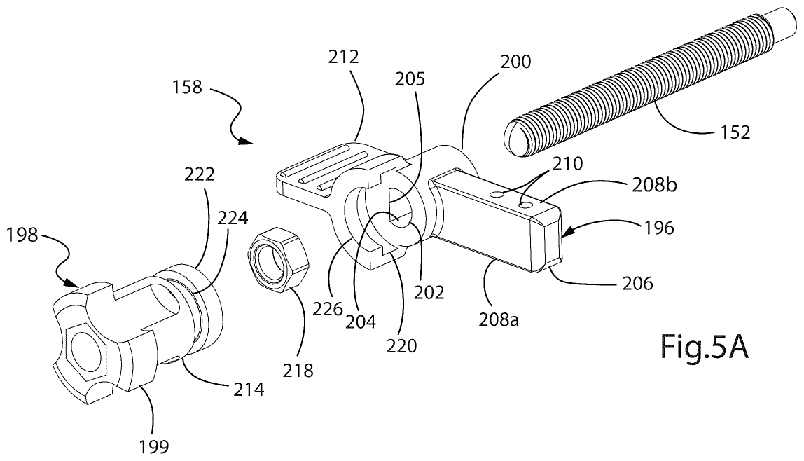

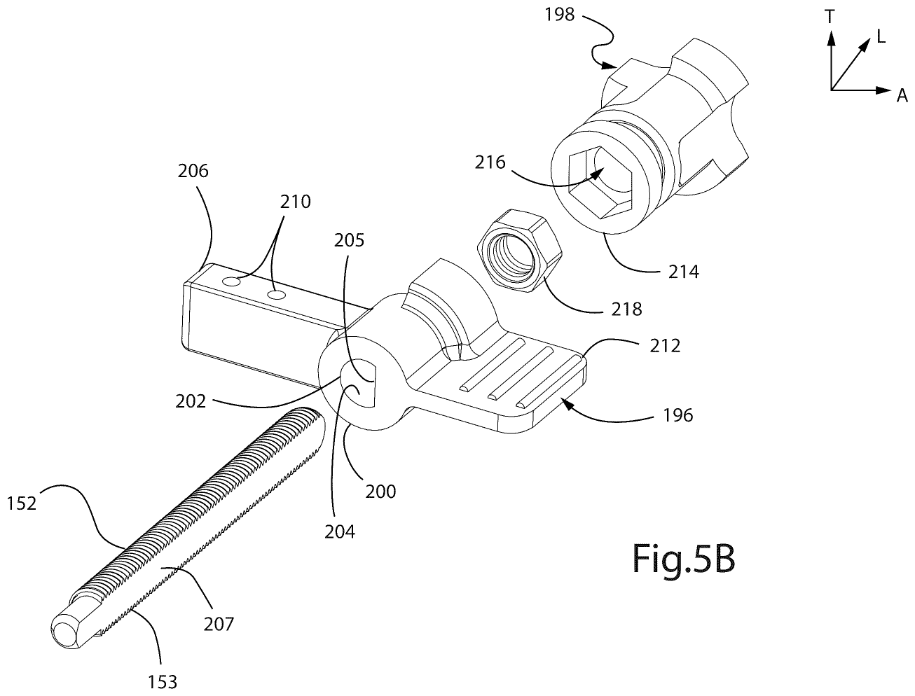

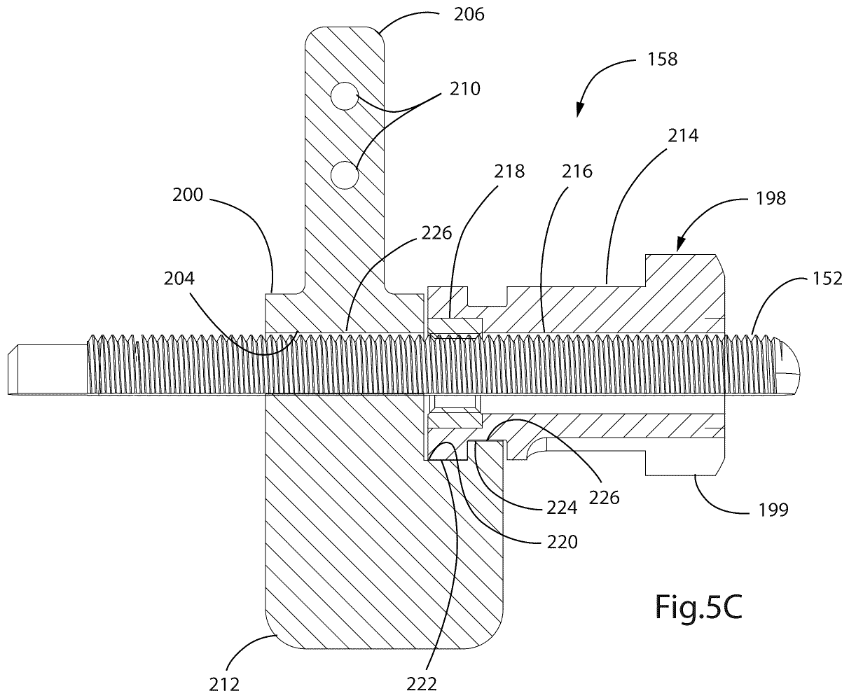

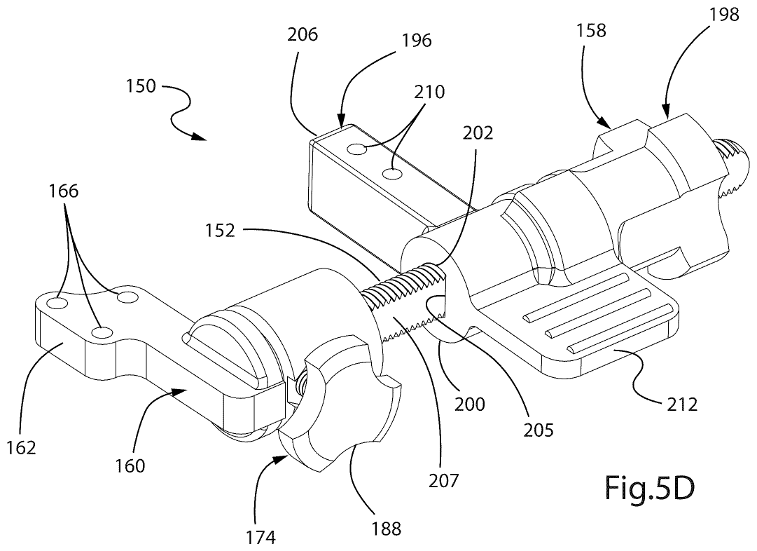

The patient's foot 100 can further include a medial cuneiform 104. The medial cuneiform 104 is generally connected with a proximal end of the metatarsal 108 (e.g., by one or more ligaments). FIGS. 1-7 illustrate methods and apparatus for correcting alignment between the medial cuneiform 104 and the metatarsal 108. In turn, proper alignment between the medial cuneiform 104 and the metatarsal 108 can correct alignment between the metatarsal 108 and the proximal phalanx 112. Accordingly, the patient's foot 100 can be manipulated from the deformed configuration 102 of the patient's foot 100 shown in FIG. 1A to a corrected configuration 103 as shown in FIG. 1B. The present disclosure relates to systems and methods for correcting the deformed configuration 102.

Moreover, the systems and methods described herein can be used more generally for correcting alignment between any two bones or bone portions of a patient's body. In this regard, the medial cuneiform 104 can be referred to as a first bone or first bone portion, and the first metatarsal 108 can be referred to as a second bone or second bone portion. It should be appreciated that the first bone and second bone can define any first and second bones of the human anatomy, wherein at least one of the first and second bones is to be rotationally and/or angularly adjusted with respect to the other of the first and second bones. It should be further appreciated that the first bone portion and the second bone portion can define any first and second portions of a single bone of the human anatomy, wherein at least one of the first and second bone portions is to be rotationally and/or angularly adjusted with respect to the other of the first and second bone portions. Thus, while the methods and apparatus described herein with respect to bunion correction, first and second bones, or the metatarsal and cuneiform, it should be appreciated that the methods and apparatus can also be applicable to other procedures involving different first and second bones or first and second bone portions, such as distal radius and flatfoot corrective surgery in some non-exhaustive examples.

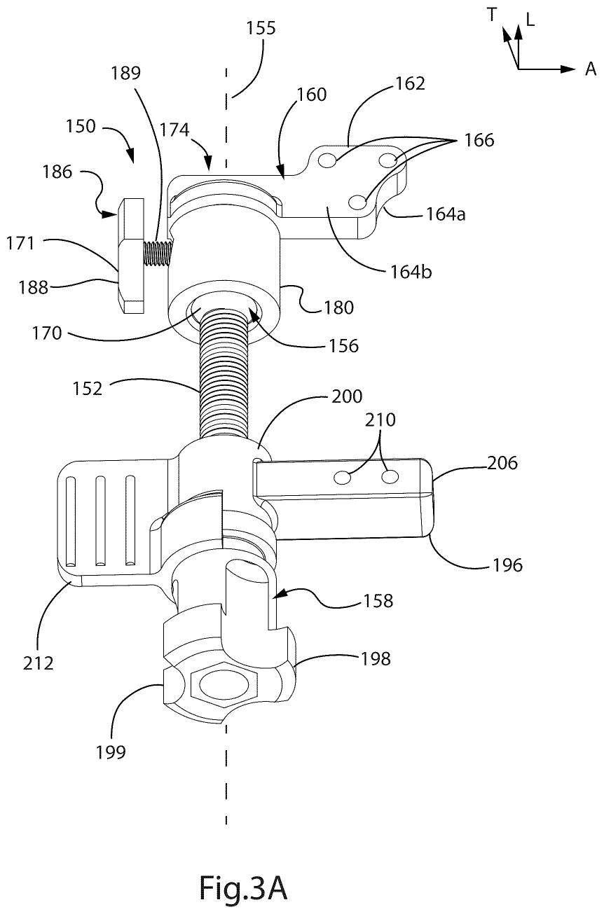

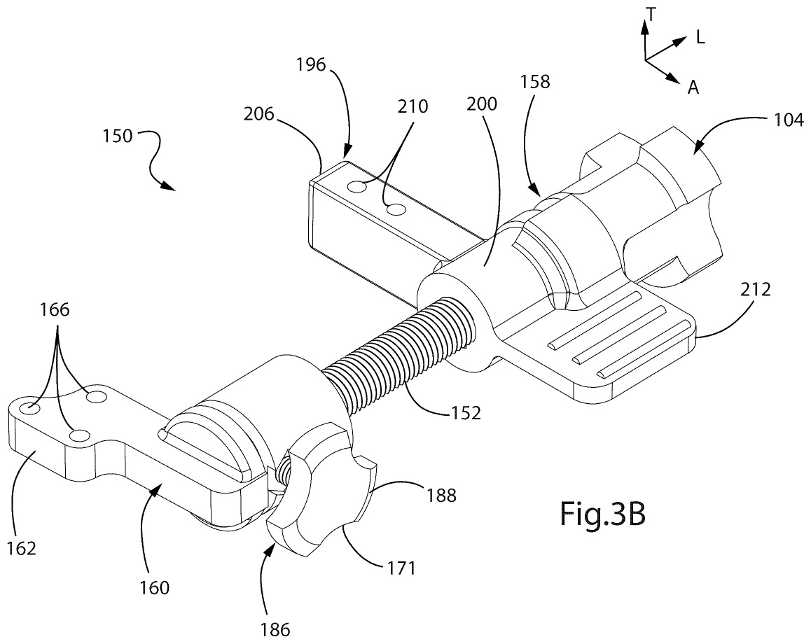

Referring now to FIGS. 3A-3B, a bone correction system 150, which can also be referred to as a bunion or hallux valgus correction system, is configured to correct alignment between first and second bone portions, which can be defined by first and second different bones or the same bone. In one example, the first and second bone portions can be defined by first and second bones of the foot, such as the first metatarsal and medial cuneiforms, respectively, as described above. The bone correction system 150 can include a first bone attachment member 160 that is configured to be temporarily secured to the medial cuneiform, and a second bone attachment member 196 that is configured to be temporarily secured to the first metatarsal. The bone correction system 150, including the components thereof, can be made of any suitable material, such as titanium, stainless steel, alloys thereof, a polymer, or any suitable alternative material as desired.

The bone correction system 150 can include an articulation joint 156 connected between the first bone attachment member and the second bone attachment member 196. The articulation joint 156 is configured to articulate the first metatarsal relative to the medial cuneiform. In particular, the articulation joint 156 can be configured to rotate the first metatarsal, about an axis of rotation relative to the medial cuneiform (also referred to as roll). Alternatively or additionally, the articulation joint 156 can be configured to angulate the first metatarsal, relative to the medial cuneiform with respect to a first view in a first plane that is defined by the anterior-posterior and the medial-lateral directions, thereby adjusting the IM angle (also referred to as IM angulation or yaw). In other words, the first view can be defined by the inferior direction. The IM angulation can therefore be about an axis of IM angulation that is perpendicular to the axis of rotation. Alternatively or additionally, still, the articulation joint 156 can be configured to angulate the first metatarsal, relative to the medial cuneiform with respect to a second view in a second plane that is defined by the anterior-posterior and superior-inferior directions (also referred to as superior-inferior angulation or pitch angulation). The pitch angulation can be about a pitch axis that is perpendicular to each of the axis of rotation and the axis of IM angulation. In other words, the second view can be defined by the medial direction. It is thus appreciated that the articulation joint 156 can be configured to manipulate the first metatarsal relative to the medial cuneiform in three orthogonal planes. In other words, when the bone correction system 150 is attached to the first metatarsal and the medial cuneiform, the first metatarsal can move in six degrees of freedom with respect to the medial cuneiform. It should be further appreciated that the universal articulation joint 156 allows for articulation about the three axes to be combined into a single motion or multiple motions as desired.

Further, the bone correction system 150 can include a translation assembly 158 that allows the first metatarsal to translate along its long axis selectively toward and away from the medial cuneiform when the bone correction system 150 is attached to the first metatarsal and the medial cuneiform. The translation assembly 158 can be disposed such that the articulation joint 156 is disposed between the first attachment member 160 and the translation assembly 158. The translation assembly 158 is configured to reduce or distract the first metatarsal relative to the medial cuneiform after the TMT joint has been resected. The TMT joint can be resected using any known system and methods in the art, such as is described in U.S. Pat. No. 11,058,546, the disclosure of which is hereby incorporated by reference as if set forth in its entirety herein.

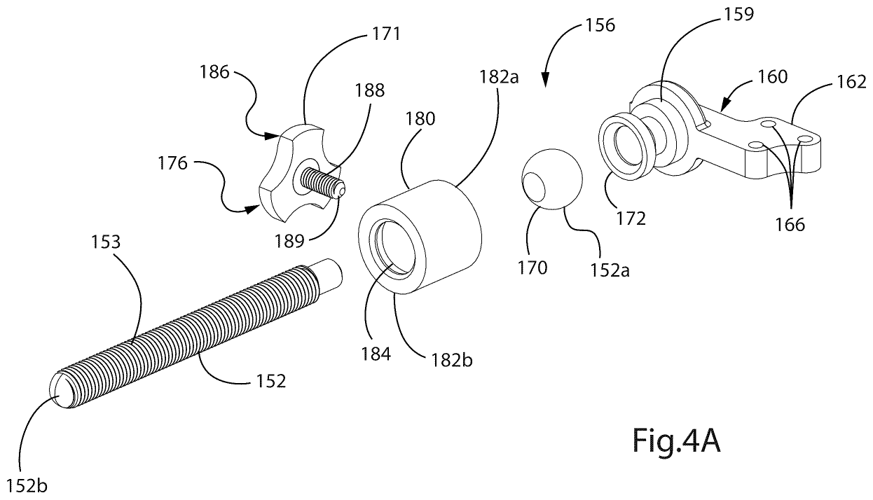

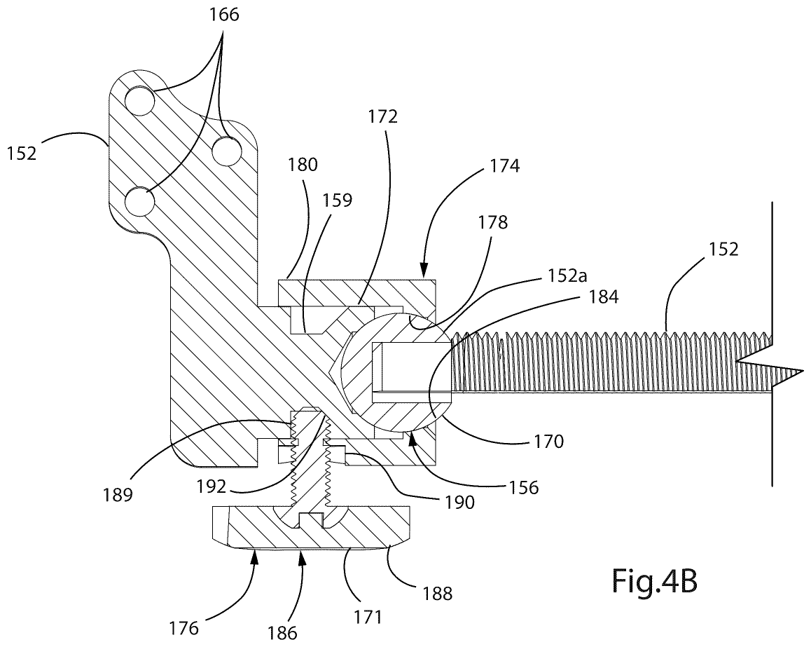

The bone correction system 150 can include a shaft 152 that extends from the articulation joint 156 to the translation assembly 158. The shaft 152 can have a first end portion 152a and a second end portion 152b opposite the first end portion 152a (see FIG. 4A). The shaft 152 can be elongate along a central shaft axis 155 that extends along a longitudinal direction L through the first end portion 152a and the second end portion 152b. The second end portion 152b can be opposite the first end portion 152a in a distal direction. Conversely, the first end portion 152a can be opposite the second end portion 152b in a proximal direction. In one example, the first end portion 152a can define a first end portion, and the second end portion 152b can define a second end portion. The shaft 152 can couple the articulation joint 156 to the translation assembly 158. For instance, the shaft 152 can be received by each of the articulation joint 156 and the translation assembly 158. In one example, the first end portion 152a of the shaft 152 can be received by the articulation joint 156, and the second end portion 152b of the shaft 152 can be received by the translation assembly 158. In this regard, each of the articulation joint 156 and the translation assembly 158 can include the shaft 152, and in particular can include the first and second end portions 152a and 152b, respectively, of the shaft 152.

Referring now to FIGS. 3A-B and 4A-4B, the first bone attachment member 160 can be configured to be removably attached to the medial cuneiform. In particular, the first bone attachment member 160 can include a coupler 159 that is configured to be coupled to the shaft 152, and a first bone attachment portion 162 that is configured to attach to the underlying medial cuneiform. Thus, reference to the coupler 159 and the first bone attachment portion 162 can apply to the first bone attachment member 160 unless otherwise indicated. The first bone attachment portion 162 can extend out from the coupler 159 along a lateral direction A that is perpendicular to the longitudinal direction L when the articulation joint 156 is in an initial unarticulated position. The initial unarticulated position of the joint 156 can refer to a position whereby the joint 156 has not undergone any of IM angulation, superior-inferior angulation, and rotation. When the articulation joint 156 undergoes IM angulation, the lateral direction can be oriented non-perpendicular with respect to the longitudinal direction L. The first bone attachment portion 162 can define an inner or bone-facing inner surface 164a, and an outer surface 164b opposite the inner surface 164a. In some examples, the first bone attachment portion 162 can be configured as a plate, whereby the inner and outer surfaces 164a-b are substantially planar along respective planes that are defined by the longitudinal direction L and the lateral direction A when the articulation joint 156 is in the initial unarticulated position.

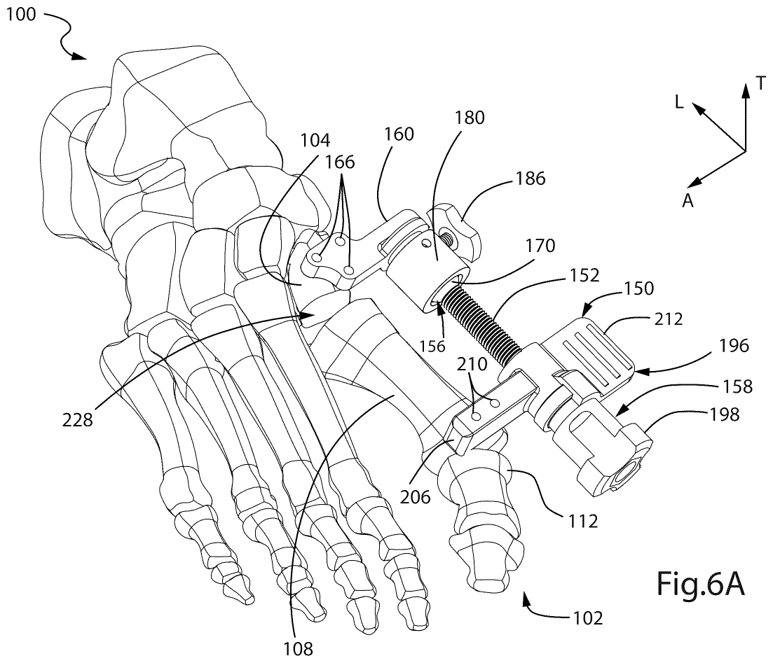

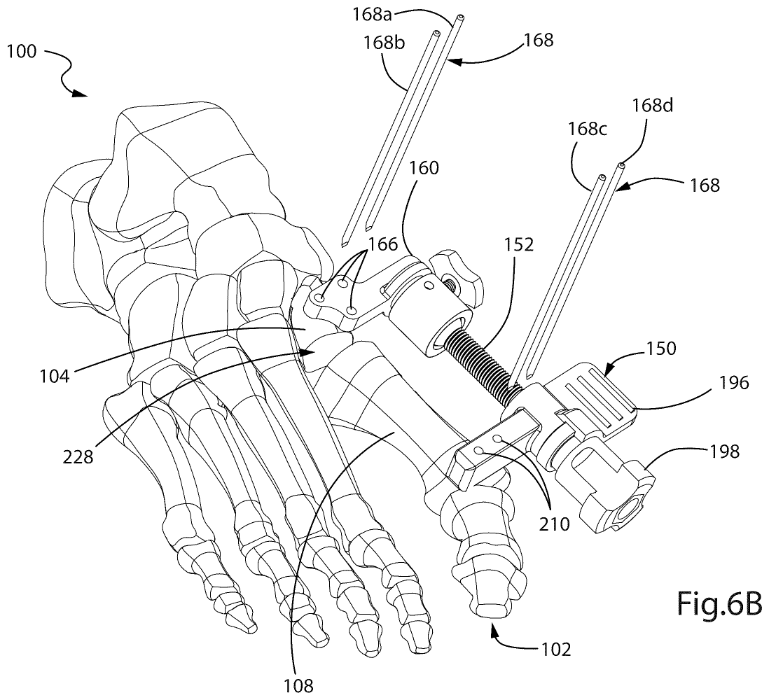

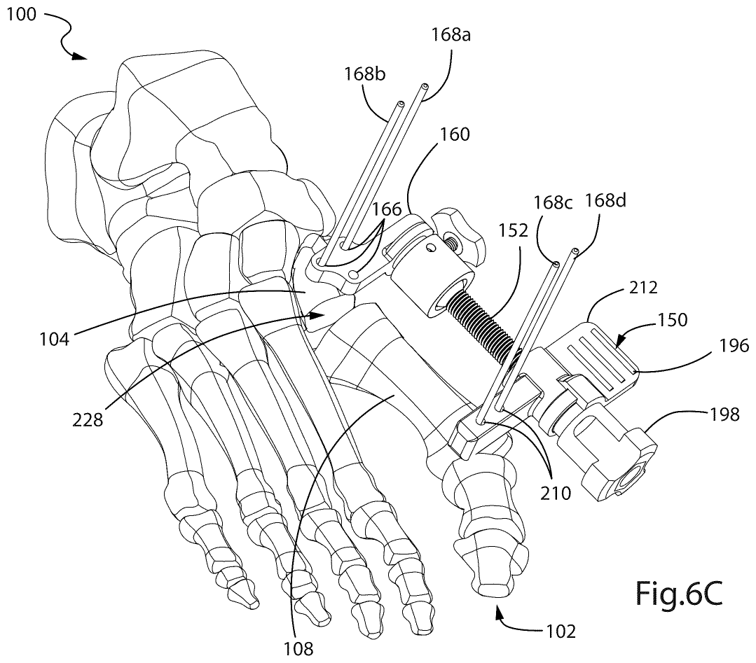

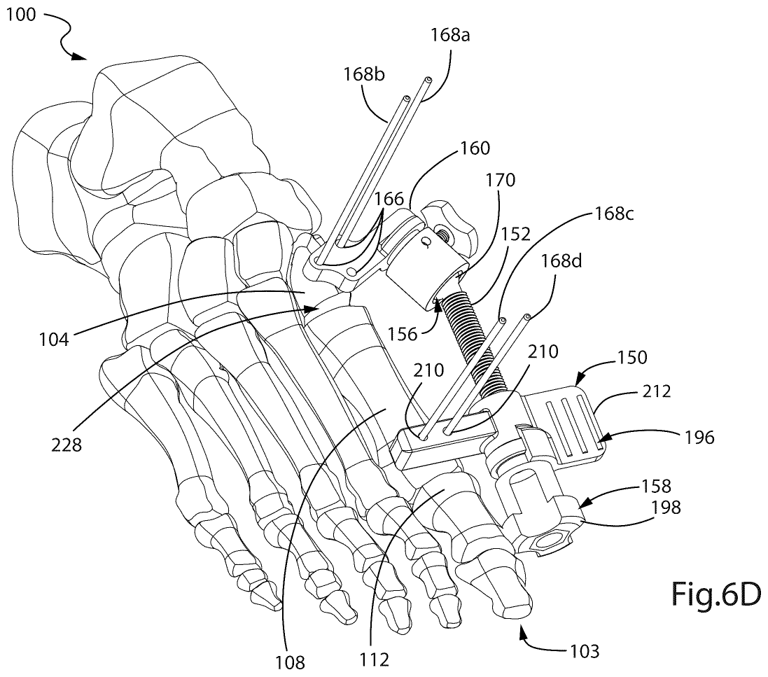

The first bone attachment member 160 can define at least one first bone fixation aperture 166 such as a plurality of first bone fixation apertures 166 that extend through the first bone attachment portion 162 from the inner surface 164a to the outer surface 164b. In one example, the first apertures 166 can be parallel to each other. It should be appreciated, of course, that the first apertures 166 can alternatively extend along different directions. Further, the first apertures 166 can all have the same diameter as each other. Alternatively, one or more of the first apertures 166 can have different diameters than the others. Each first aperture 166 can be sized and configured to receive a respective bone fixation member 168 (see FIG. 6C) that extends through the first aperture 166 and into the medial cuneiform 104. The apertures 166 can thus be spaced from each other a desired distance in one or more desired directions based on a size and shape of the medial cuneiform. A bone correction assembly can include the bone correction system 150 and the bone fixation members 168.

The bone fixation members 168 can be temporary bone fixation members that is removed prior to completion of the surgical procedure. Each of the bone fixation members 168 can be configured as a pin, a Kirschner wire (K-wire), or ay suitable alternative bone fastener as desired. The first bone attachment member 160 can include any number of first apertures 166 as desired so as to provide flexibility in aligning at least one of the first apertures 166 with the medial cuneiform 104 (see FIGS. 6A-C). The central axis of the shaft 152 can be offset with respect to the apertures 166 by an offset distance in an offset direction. The offset direction can be the lateral direction A when the articulation joint 156 is in the initial position, or if the articulation joint 156 has not undergone IM articulation. The offset direction can be angularly offset with respect to the lateral direction A when the articulation joint 156 has undergone IM angulation. It is envisioned in some examples that at least one of the first apertures 166 can be aligned with the central shaft axis 155, such that the at least one of the first apertures is not offset from the central shaft axis 155.

With continuing reference to FIGS. 3A-4B, the first bone attachment member 160 can further include a coupler 159 that forms part of the articulation joint 156. The coupler 159 can be monolithic with the first bone attachment portion 162, or can be separate from and attached to the first bone attachment portion 162. In one example, the coupler 159 can be configured to be coupled to the shaft 152 so as to define the articulation joint 156. For instance, the coupler 159 can be coupled to the first end portion 152a of the shaft 152. The shaft 152 can be configured to articulate with the coupler 159 (and thus the first bone attachment portion 162) when the articulation joint 156 is in an unlocked configuration described in more detail below. As will be described below, the bone correction system can be configured to be attached to the medial cuneiform and the first metatarsal, such that articulation of the shaft 152 relative to the first bone attachment portion 162 causes the first metatarsal to correspondingly articulate with respect to the medial cuneiform.

For instance, the first bone attachment member 160 and the shaft 152 can be configured to rotate with respect to each other about an axis of rotation. In one example, when the articulation joint is in an un-angulated position, the central axis of rotation can be defined by the central shaft axis 155 of the shaft 152. The central shaft axis 155 can be oriented along a direction that is substantially parallel to the long axis of the first metatarsal 108 when the articulation joint 156 is in an initial unarticulated position (see FIG. 6C). Further, either or both of the first bone attachment member 160 and the shaft 152 can be configured to angulate with respect to the other of the first bone attachment member 160 and the shaft 152 about an axis of IM angulation. The axis of IM angulation can be perpendicular to the axis of rotation, and also perpendicular to the lateral direction A when the articulation joint 156 is in the unangulated position. The unangulated position of the joint 156 can refer to a position whereby the joint 156 has not undergone any of IM angulation and superior-inferior angulation. The axis of IM angulation can be oriented in a transverse direction T that is perpendicular to each of the lateral direction A and the longitudinal direction L when the articulation joint is in the initial unarticulated position. The lateral direction A and the longitudinal direction L define an angle that varies as either or both of the first bone attachment member 160 and the shaft 152 angulates with respect to the other of the first bone attachment member 160 and the shaft 152 about an axis of IM angulation. As will be appreciated from the description below, the IM angulation of the first bone attachment member 160 can cause the first metatarsal to correspondingly selectively angulate toward and away from the second metatarsal, thereby decreasing or increasing, respectively, the IM angle.

In one example, the first end portion 152a of the shaft 152 can define a head 170. The head 170 can be spherical in one example. In some examples, the head 170 is an enlarged head having a larger diameter than the shaft 152. In some examples, the head 170 can be smooth and unthreaded. Thus, the articulation joint 156 can be a universal joint in some examples. Alternatively, the articulation joint 156 can include guidance features to guide one or more up to all of IM angulation, superior-inferior angulation, and rotation. For instance, in one example the articulation joint can allow for IM angulation and rotation, but not superior-inferior angulation. Thus, the articulation joint 156 can allow for the manipulation of the shaft 152 with respect to the first bone attachment member 160 in at least two orthogonal planes, such as in three orthogonal planes. In one example, the shaft 152 can include a threaded shaft body 153, and the head 170 can extend from the threaded shaft body 153. In one example, the head 170 can be attached to the threaded shaft body 153 at the first end portion 152a. Alternatively, the head 170 can be monolithic with the threaded shaft body 153. As will now be described, the articulation joint 156 is configured to move between an unlocked configuration and a locked configuration. When the articulation joint 156 is in the unlocked configuration, the first bone attachment portion 162 and the shaft 152 are able to articulate with respect to each other in the manner described above. When the articulation joint 156 is in the locked configuration, the first bone attachment portion 162 and the shaft 152 are unable to articulate with respect to each other.

In one example, the coupler 159, and thus the first bone attachment member 160, can include a collet 172 that is configured to engage the enlarged head 170. Thus, the articulation joint 156 can include the collet 172. When the articulation joint 156 is in the unlocked configuration, the collet 172 does not prevent relative articulation between the first bone attachment portion 162 and the shaft 152. In one example, the head 170 of the shaft 152 is able to articulate with respect to the collet 172. When the articulation joint 156 is in the locked configuration, the collet 172 can bear against the head 170 of the shaft 152 so as to prevent relative articulation between the first bone attachment portion 162 and the shaft 152.

The bone correction system 150 can include an articulation assembly 174 that includes the articulation joint 156 and a lock 176 that is configured to iterate the articulation joint 156 between the locked and unlocked configurations. While the lock 176 can be configured in accordance with any suitable embodiment, in one example the lock 176 can include a seat 178 and the collet 172. The seat 178 can be configured to cooperate with the collet 172 such that the head 170 of the shaft 152 is disposed between the collet 172 and the seat 178. When the lock 176 is in the unlocked position, articulation joint 156 is in the unlocked configuration as described above, and the head 170 can articulate with respect to the collet 172 and the seat 178. In this regard, an external surface of the head 170 can define an articulation surface that articulates within the joint 156. When the lock 176 is in the locked position, the collet 172 and the seat 178 capture the head 170 of the shaft 152 therebetween, thereby preventing the head 170 and collet 172 from articulating with respect to each other. Thus, when the lock 176 is in the locked position, the articulation joint 156 is in the locked configuration.

In one example, the seat 178 can be defined by a sleeve 180 that surrounds the collet 172. At least a portion of the head 170 can extend into the sleeve 180 in the first direction. In one example, the sleeve 180 can have a first end portion 182a that surrounds at least a portion of the collet 172, and a second end portion 182b that surrounds at least a portion of the head 170 of the shaft 152. The sleeve 180 can be annular, such as cylindrical, in one example. The sleeve 180 can include a flange 184 that extends inward from the second portion 182b so as to be aligned with the head, such that the flange 184 is configured to bear against the head 170. The flange 184 can define the seat 178.

The lock 176 can include an actuator 186 that is configured to iterate between an engaged position and a disengaged position. In the engaged position, the actuator 186 causes the collet 172 and the seat 178 to bear against the head 170 of the shaft 152 with sufficient compression locking force to preventing the shaft 152 and the first bone attachment member 160 from articulating with respect to each other. Thus, iterating the actuator 186 in the engaged position moves the lock 176 to the locked position, which in-turn causes the articulation joint 156 to be in the locked configuration. When the actuator 186 is in the disengaged position, at least a portion of the locking force is removed, such that the shaft 152 and the first bone attachment member 160 are configured to articulate with respect to each other in the manner described above. Thus, when the actuator 186 is in the disengaged position, the lock 176 is in the unlocked position, which in-turn causes the articulation joint 156 to be in the unlocked configuration.

In one example, the actuator 186 can be configured as a set screw 188 that extends through an aperture 190 of the sleeve 180. For instance, the set screw 188 can have a shaft 189 that is threadedly mated with the sleeve 180 as it extends through the aperture 190. Accordingly, rotation of the set screw 188 in a locking direction causes the shaft 189 of the set screw 188 to translate toward the collet 172 to the engaged position. Rotation of the set screw 188 in an unlocking direction opposite the locking direction causes the shaft 189 of the set screw 188 to translate away from the collet 172 to the disengaged position. The set screw 188 can include an ergonomic handle 171 that can be gripped by the medical professional to rotate the set screw 188 as desired. The shaft 189 can extend from the handle 171. The shaft 189 can be configured to ride along a ramped cam surface 192 of the collet 172 as the set screw 188 moves to the engaged position. The ramped surface 192 of the collet 172 can be defined by an external surface of the collet 172. As the shaft 189 rides along the ramped surface 192, the shaft 189 urges the collet 172 to move toward the head 170 of the shaft 152. The collet 172, in turn, urges the enlarged head 170 against the sleeve 180, and in particular against the seat 178. Thus, the articulation joint 156 is in the locked configuration, whereby the head 170 is captured between the collet 172 and the seat 178 with sufficient locking force such that the shaft 152 and the first bone attachment portion 162 are prevented from articulating with respect to each other.

When the set screw 188 rotates in the unlocking direction, the locking force from the set screw 188 is removed from the ramped surface 192, thereby removing the locking force from the collet 172. It should be appreciated that the set screw 188 can be moved a sufficient distance to the disengaged position whereby all of the compression force previously applied to the shaft head 170 from the collet 172 and the sleeve 180 is removed. Thus, the shaft 152 and the first bone attachment portion 162 can articulate freely with respect to each other at the articulation joint 156 in the manner described above. In other examples, the set screw 188 can be moved a sufficient distance so that the locking force is removed, but a retention force is applied by the lock 176 (and in particular by the collet 172 and the sleeve 180) that prevents relative articulation between the shaft 152 and the first bone attachment portion 162 absent an articulation force sufficient to overcome the retention force. An articulation force can be applied to either or both of the shaft 152 and the first bone attachment portion 162 sufficient to overcome the retention force and articulate either or both of the shaft 152 and the first bone attachment portion 162 relative to the other of the shaft 152 and the first bone attachment portion 162 in the manner described above. It should be appreciated, of course, that any suitable alternatively locking system is envisioned to selectively lock the first bone attachment member 160 to, and unlock the first bone attachment member from, the shaft 152 with respect to relative rotation and angulation.

Referring now to FIGS. 3A-B and 5A-D, the bone correction system 150 can also include the translation assembly 158 that is configured to be removably attached to the first metatarsal and translate along the shaft 152. In particular, the translation assembly 158 has a second bone attachment member 196 that is configured to be supported by the shaft 152 and removably attached to the first metatarsal. The second bone attachment member 196 can thus be configured to translate selectively toward and away from the first bone attachment member 160 along the shaft 152. Movement of the second bone attachment member 196 assembly can selectively decrease and increase a distance between the medial cuneiform and the first metatarsal, for instance along a direction defined by the central axis of the shaft 152. Otherwise stated, movement of the second bone attachment member 196 along the shaft 152 can distract or reduce a gap between the medial cuneiform and the first metatarsal. The translation assembly 158 can include a translation actuator 198 that is configured to drive the second bone attachment member 196 to translate along the shaft 152. It should be recognized that while the first bone attachment member 160 is attached to the medial cuneiform and the second bone attachment member 196 is attached to the first metatarsal in some examples, in other examples, the first bone attachment member 160 can be temporarily attached to the first metatarsal and the second bone attachment member 196 can be temporarily attached to the medial cuneiform.

Referring again to FIGS. 3A-B and 5A-D, the second bone attachment member 196 can include a second bone attachment member body 200 and a shaft receiving aperture 202 that extends through the body 200 along the longitudinal direction L. The shaft receiving aperture 202 is sized to receive the shaft 152, such that the shaft 152 extends into or through the shaft receiving aperture 202. The shaft receiving aperture 202 can be smooth so that it does not threadedly mate with the shaft 152. Thus, the second bone attachment member 196 can translate along the shaft 152 without rotation about the shaft 152. Further, the second bone attachment member 196 and the shaft 152 can be keyed so as to prevent rotation of the second bone attachment member about the shaft 152. For example, an internal surface 204 of the body 200 defines the shaft receiving aperture 202. A first portion of the internal surface 204 can be cylindrical in cross-section, and a second portion of the internal surface 204 can be non-cylindrical. In one example, the second portion of the internal surface 204 can be substantially planar so as to define a flat 205. Similarly, a first portion of the shaft 152 can be cylindrical in cross-section, and a second portion of the shaft 152 in cross-section can be non-cylindrical. For instance, the second portion of the shaft 152 can be substantially planar so as to define a flat 207. Thus, the shaft 152 geometry corresponds to the geometry of the shaft receiving aperture 202, such that the shaft 152 can be received in the shaft receiving aperture 202 in only one relative orientation. Further, the non-circular cross-sections of the shaft receiving aperture 202 and the shaft 152 prevent the second bone attachment member 196 from rotating about the shaft 152 as it translates along the shaft 152.

Thus, in one example, translation of the second bone attachment member 196 along the shaft 152 can be a pure translation. In other examples, the second bone attachment member 196 can be rotatable about the shaft 152. For instance, the shaft receiving aperture 202 and the shaft 152 can be cylindrical, and the internal surface 204 can threadedly mate with the shaft 152. Thus, rotation of the shaft 152 while preventing rotation of the second bone attachment member 196 can cause the second bone attachment member 196 to selectively translate toward and away from the first bone attachment member 160. In this regard, the second bone attachment member 196 can be referred to as a traveler that selectively travels along the shaft 152 toward and away from the first bone attachment member 160.

With continuing reference to FIGS. 3A-B and 5A-D, the second bone attachment member 196 can include a second bone attachment portion 206 that is configured to be removably attached to the first metatarsal. The second bone attachment portion 206 can extend from the body 200 along the lateral direction A when the articulation joint 156 is in the initial unarticulated position. At least a portion of the first bone attachment portion 162 and at least a portion of the second bone attachment portion 206 can be aligned with each other along the longitudinal direction L when the articulation joint 156 is in the initial unarticulated position. The second bone attachment portion 206 can define an inner or bone-facing inner surface 208a, and an outer surface 208b opposite the inner surface 208a. In some examples, the second bone attachment portion 206 can be configured as a plate, whereby the inner and outer surfaces 208a-b are substantially planar along respective planes that are defined by the longitudinal direction L and the lateral direction A when the articulation joint 156 is in the initial unarticulated position.

The second bone attachment member 196 can define at least one second bone fixation aperture 210 such as a plurality of second bone fixation apertures 210 that extend through the second bone attachment portion 206 from the inner surface 208a to the outer surface 208b. In one example, the second apertures 210 can be parallel to each other. It should be appreciated, of course, that the second apertures 210 can alternatively extend along different directions. Further, the second apertures 210 can all have the same diameter as each other. Alternatively, one or more of the second apertures 210 can have different diameters than the others. Each second aperture 210 can be sized and configured to receive a respective one of the bone fixation members 168 (see FIG. 6C) that extends through the second aperture 210 and into the first metatarsal 108. The second bone fixation apertures 210 can thus be spaced from each other a desired distance in one or more desired directions based on a size and shape of the first metatarsal 108. The second bone attachment member 196 can include any number of second apertures 210 as desired so as to provide flexibility in aligning at least one of the second apertures 210 with the first metatarsal 108 (see FIGS. 4A-C). The central axis of the shaft 152 can be offset with respect to the apertures 166 by an offset distance in the offset direction. The offset direction can be the lateral direction A when the articulation joint 156 is in the initial position, or if the articulation joint 156 has not undergone IM articulation. The offset direction can be angularly offset with respect to the lateral direction A when the articulation joint 156 has undergone IM angulation. It is envisioned in some examples that at least one of the second apertures 210 can be aligned with the central shaft axis 155, such that the at least one of the second apertures is not offset from the central shaft axis 155. The second apertures 210 can be aligned with the first apertures 166, or can be angularly offset with respect to the first apertures. Further, the second apertures 210 can be sized equal to the first apertures 166, or can be sized differently as desired.

The second bone attachment member 196 can further include a grip tab 212 that extends out from the body 200. In one example, the grip tab 212 can extend from the body 200 along the lateral direction A when the articulation joint is in the initial or unarticulated position. The grip tab 212 can extend from the body 200 along a direction opposite the second bone attachment portion 206. Thus, the second bone attachment portion 206 can extend from the body 200 along a first direction, and the grip tab 212 can extend from the body 200 along a second direction that is opposite the first direction. Each of the first and second directions can be oriented along the lateral direction A when the articulation joint 156 is in the initial unarticulated position. The grip tab 212 can have a textured outer surface as desired so as to facilitate reliable gripping of the grip tab 212 by the medical professional during use. During use, manipulation of the grip tab 212 can cause the second bone attachment member 196 and the shaft 152 to articulate about the articulation joint 156. The grip tab 212 can also be gripped by the medical professional to provide counter-torque to torsional forces that are applied to the shaft 152 during rotation of the actuator 198. The actuator 198 can include an ergonomic handle 199 that can be configured to be easily gripped by the medical professional, and apply a torsional force to the handle 199 that facilitates rotation of the actuator 198. The counter-torque can prevent inadvertent rotation of the shaft 152 about its axis 155 at the articulation joint 156 as the actuator 198 is rotated. It should be appreciated that the articulation joint 156 is connected between the first bone attachment member 160 and the second bone attachment member 196, meaning that the first and second bone attachment members 160 and 196 can articulate with respect to each other about the articulation joint 156.

With continuing reference to FIGS. 3A-B and 5A-D, the actuator 198 can be threadedly mated with the shaft 152. Further, the actuator 198 can be rotatable about the shaft axis 155, which causes the actuator 198 to travel along the shaft 152. The actuator 198 can be translatably coupled to the second bone attachment member 196. Rotation of the actuator 198 with respect to the shaft 152 in a first direction of rotation causes the actuator 198, and thus the second bone attachment member 196, to travel along the shaft 152 toward the first bone attachment member 160. Rotation of the actuator 198 with respect to the shaft 152 in a second direction of rotation opposite the first direction of rotation causes the actuator 198, and thus the second bone attachment member 196, to travel along the shaft 152 away from the first bone attachment member 160. The first and second directions of rotation can be about an axis of rotation. The axis of rotation can be defined by the central shaft axis 155 of the shaft 152.

The actuator 198 will now be described in more detail. In particular, the actuator 198 can have an actuator body 214 and a bore 216 that extends through the actuator body 214 along the longitudinal direction L. The bore 216 is sized to receive the shaft 152. Further, the actuator body 214 can receive a threaded member 218, such as a nut, that is disposed in the bore 216 and is positionally fixed with respect to the actuator body 214. The threaded member 218 can threadedly mate with the shaft 152 such that rotation of the actuator body 214, and thus the actuator 198, with respect to the shaft 152 causes the actuator 198 to travel along the shaft 152 away from or toward the first bone attachment member 160. In other example, the bore 216 can be a threaded bore, such that the actuator body 214 is threadedly mated with the shaft 152. In still other examples, either or both of the actuator 198 and the second bone attachment member 196 can define a ratchet with the shaft 152, so as to ratchet over the threads of the shaft 152 during translation along the shaft 152. In each example, the second bone attachment member 196 is configured to translate along the shaft 152. When the first bone attachment member 160 is attached to the medial cuneiform, and the second bone attachment member 196 is attached to the first metatarsal, translation and articulation of the second bone attachment member 196 with respect to the first bone attachment member 160 translates and articulates the first metatarsal with respect to the medial cuneiform.

With continuing reference to FIGS. 3A-B and 5A-D, the actuator 198 can be rotatable with respect to the second bone attachment member 196 in each of the first and second directions of rotation, and translatably fixed to the second bone attachment member 196. Thus, selection rotation of the actuator 198 in the first and second directions of rotation occurs with respect to the second bone attachment member 196. In particular, the second bone attachment member 196 can define a channel 220 that extends about the axis of rotation. The actuator 198 can include a rail 222 that extends from the actuator body 214 and into the channel 220. The rail 222 translates within and along the channel 220 as the actuator 198 rotates with respect to the second bone attachment member 196. The rail 222 can occupy a substantial entirety of the channel 220 along the longitudinal direction L, so as to fix the second bone attachment member 196 to the actuator 198 with respect to travel along the shaft 152. In other words, engagement of the rail 222 with the second bone attachment member 196 in the channel 220 can second bone attachment member 196 to travel with the actuator 198 both toward and away from the first bone attachment member 160. Alternatively or additionally, the actuator 198 can define a channel 224 that extends about the axis of rotation. The second bone attachment member 196 can include a rail 226 that extends into the channel 224. The rail 226 translates within and along the channel 224 as the actuator 198 rotates with respect to the second bone attachment member 196. The rail 226 can occupy a substantial entirety of the channel 224 along the longitudinal direction L, so as to fix the second bone attachment member 196 to the actuator 198 with respect to travel along the shaft 152. In other words, engagement of the rail 226 with the actuator 198 in the channel 224 can second bone attachment member 196 to travel with the actuator 198 both toward and away from the first bone attachment member 160.

A method of correcting alignment between the medial cuneiform 104 and the first metatarsal 108 will now be described with respect to FIGS. 6A-6D. Depending on a planned corrected configuration of the first metatarsal 108 with respect to the medial cuneiform 104, it may be desired to remove material from one or both of the distal end of the medial cuneiform 104 and the proximal end of the first metatarsal 108. As shown in FIG. 6A, the distal end of the medial cuneiform 104 and the proximal end of the first metatarsal 108 can be resected, thereby removing the TMT joint and enlarging a gap 228 between the medical cuneiform 104 and the first metatarsal 108. The resection can be performed in accordance with any suitable method as desired, for instance as described in U.S. Pat. No. 11,058,546, the disclosure of which is hereby incorporated by reference as if set forth in its entirety herein. The resection can increase the mobility of the first metatarsal 108 with respect to the medial cuneiform 104. In particular, the first metatarsal 108 can be translated with respect to the medial cuneiform 104, can undergo IM angulation with respect to the medial cuneiform 104, can rotate about its long axis with respect to the medial cuneiform 104 and can undergo superior-inferior angulation with respect to the medial cuneiform 104.

The bone correction system 150 can be attached to the first metatarsal 108 and the medial cuneiform 104 either before or after the resection. In particular, as shown at FIGS. 6A, the bone correction system 150 is aligned for fixation with the patient's foot 100, and in particular with the medial cuneiform 104 and the first metatarsal 108. In particular, the first bone attachment member 160 is aligned with the medial cuneiform 104 such that a central axis of at least one of the first bone fixation apertures 166 is aligned with the medial cuneiform 104. In one example, a pair of the first apertures 166 can be aligned with the medial cuneiform. The second bone attachment member 196 is aligned with the first metatarsal 108 such that a central axis of at least one of the second bone fixation apertures 210 is aligned with the first metatarsal 108. In one example, a pair of the second apertures 210 can be aligned with the first metatarsal 108.

As shown in FIGS. 6B-6C, a plurality of bone fixation members 168 can be driven through respective first and second bone fixation apertures 166 and 210 and into the medial cuneiform 104 and the first metatarsal 108, respectively. For instance, a first bone fixation member 168a, such as a first K-wire, can be driven through a first one of the first bone fixation apertures 166 and into the medial cuneiform 104. A second bone fixation member 168b, such as a second K-wire, can be driven through a second one of the first bone fixation apertures 166 and into the medial cuneiform 104. A third bone fixation member 168c, such as a third K-wire, can be driven through a first one of the second bone fixation apertures 210 and into the first metatarsal 108. A fourth bone fixation member 168d, such as a fourth K-wire, can be driven through a second one of the second bone fixation apertures 210 and into the first metatarsal 108. It should be appreciated that the bone fixation members 168a-d can be driven into the respective bone in any order as desired. Further, the bone fixation members 168a-d can be driven into the respective bone before or after resection of the medial cuneiform 104 and/or the first metatarsal 108.

The first and second bone fixation members 168a and 168b can be parallel with each other, based on the parallel first bone fixation apertures 166. The third and fourth bone fixation members 168c and 168d can be parallel with each other, based on the parallel second bone fixation apertures 210. The first and second bone fixation members 168a-b can be parallel or non-parallel with respect to the third and fourth bone fixation members 168c-d. The diameters of the bone fixation members 168 can be sized according the diameters of the respective bone fixation apertures 166 and 210 to ensure accurate insertion angles into the respective medial cuneiform 104 and first metatarsal 108. The diameters of the first bone fixation apertures 166 can be equal to, greater than, or less than, the diameters of the second bone fixation apertures 210.

The bone correction system 150 can be in a first position when the bone fixation members are first attached to the medial cuneiform 104 and the first metatarsal 108. In one example, the first position can be defined by the initial or unarticulated position of the bone correction system 150. Alternatively, in the first position can be articulated and/or translated with respect to the initial or unarticulated position. For example, to achieve the first position, it may be desired to articulate and/or translate the second bone attachment member 196 relative to the first bone fixation member 160 in order to align a desired number of the bone fixation apertures 166 and 210 with the respective underlying medial cuneiform 104 and first metatarsal 108 for fixation. The articulation joint 156 can be in the locked or unlocked configuration when attaching the bone correction system 150 to the patient's foot 100. That is, the actuator 186 can be in the engaged position or in the disengaged position, as described above with respect to FIGS. 4A-4B, when attaching the bone correction system 150 to the patient's foot 100.

Referring now to FIGS. 6C-6D, and also with reference to FIGS. 3A-5D, after the bone correction system 150 has been attached to the patient's foot 100, the bone correction system 150 can be manipulated so as to move the first metatarsal 108 relative to the medial cuneiform 104 to the corrected configuration 103 of the patient's foot 100. For instance, the articulation joint 156 can be moved to, or remain in, the unlocked configuration. In one example, the shaft 152 and thus the second bone attachment member 196 is freely movable with respect to the first bone attachment member 160. Alternatively, as described above, the articulation joint 156 can provide a retention force that maintains the articular position of the shaft 152 and the second bone attachment member 196 relative to the first bone attachment member 160. However, a sufficient external articulation force can be applied to either or both of the shaft 152 and the first bone attachment portion 162 sufficient to overcome the retention force and articulate either or both of the shaft 152 and the first bone attachment portion 162 relative to the other of the shaft 152 and the first bone attachment portion 162. It is appreciated that articulation of the shaft 152 relative to the first bone attachment member 160 causes the second bone attachment member 196 to similarly articulate with respect to the first bone attachment member 160. Because the second bone attachment member 196 is attached to the first metatarsal 108, and the first bone attachment member 160 is attached to the medial cuneiform 104, movement (i.e., articulation and translation) of the second bone attachment member 196 relative to the first bone attachment member 160 causes the first metatarsal 108 to similarly move relative to the medial cuneiform 104. Thus, the bone correction system 150 can move the first metatarsal 108 until the patient's foot 100 is iterated from the deformed configuration 102 shown in FIGS. 6A-6C to the corrected configuration 103 shown in FIG. 6C. In the corrected configuration 103, the first metatarsal 108 can be articulated relative to the medial cuneiform 104 until the first metatarsal is generally aligned with (or more aligned with than in the deformed configuration 102) the proximal phalanx 112 of the great toc.

The articulation joint 156 can be manipulated in one example by grasping the shaft 152 or the second bone attachment member 196, and providing a sufficient force so as to cause the shaft 152 and the second bone attachment member 196 to articulate about the articulation joint 156 with respect to the first bone attachment member 160. In one example, the first grip tab 212 of the second bone attachment member 196 can be grasped by the medical professional to articulate the second bone attachment member 196. As described above, articular movement of the second bone attachment member 196 about the articulation joint 156 causes the shaft 152 to articulate with respect to the first bone attachment member 160.

Thus, rotation of the second bone attachment member 196 about the axis of rotation similarly causes the first metatarsal 108 to rotate or revolve about the axis of rotation with respect to the medial cuneiform. The bone correction system 150 can allow for 360 degrees of rotation of the second bone attachment member 196 relative to the first bone attachment member about the axis of rotation. The shaft 152, and thus the second bone attachment member 196, and thus the first metatarsal 108, can be angulated about the articulation joint 156 relative to the medial cuneiform closer to the second metatarsal or further from the second metatarsal to define a desired IM angle. In typical bunion correction procedures, the first metatarsal 108 will be angulated toward the second metatarsal to reduce the IM angle to an acceptable level, which can be less than 11 degrees, such as less than 9 degrees in some examples. The shaft 152, and thus the second bone attachment member 196, and thus the first metatarsal 108, can be angulated about the articulation joint 156 relative to the medial cuneiform 104 superiorly or inferiorly as desired, thereby adjusting the pitch of the first metatarsal 108. It should be appreciated that second bone attachment member 196, and thus the first metatarsal 108, can be angulated to adjust the IM angle and the pitch individually or in combination. The second bone attachment member 196 can angulated with respect to the first bone attachment member 160 until the shaft body 153 abuts the sleeve 180 which prevents further angulation. Advantageously, the bone correction system 150 can achieve up to 45 degrees of angulation. Thus, the bone correction system 150 can be attached to the first metatarsal 108 and the medial cuneiform 104 in the manner described above even in instances of severe hallux valgus deformity in which the first metatarsal is severely angulated with respect to the medial cuneiform. It is recognized that 87% of HV are misaligned in three dimensions. Advantageously, the articulation joint 156 is configured to articulate the first metatarsal 108 relative to the medial cuneiform 104 in the three dimensions.

The bone correction system 150 can further include the translation assembly 158 that can be actuated in the manner described above, thereby adjusting a distance from the second bone attachment member 196 to the first bone attachment member 160, and thus from the first metatarsal 108 to the medial cuneiform 104, along a straight line. The straight line can be defined by the central shaft axis 155 of the shaft 152 (see FIG. 3A). In one example, the second bone attachment member 196 can be brought toward the first bone attachment member 160, thereby bringing the first metatarsal 108 abuts the medial cuneiform 104. That is, the first metatarsal 108 and the medial cuneiform are closer together in the corrected configuration 103 with respect to the deformed configuration 102. In one example, the first metatarsal 108 and the medial cuneiform can be brought closer together until the first metatarsal 108 abuts the medial cuneiform 104 in the corrected configuration 103. This abutment can promote the union or fusion of the metatarsal 108 with the medial cuneiform 104 during healing.

It should thus be appreciated that the bone correction system 150 is iterated from the first position when attached to the bones in the deformed configuration 102, to a final position when attached to the bones in the corrected configuration 103. Once the bone correction system 150 is in the final position, the articulation joint 156 can be iterated to the locked configuration, such that the bone correction system 150 remains in the final position with the patient's foot in the corrected configuration 103.

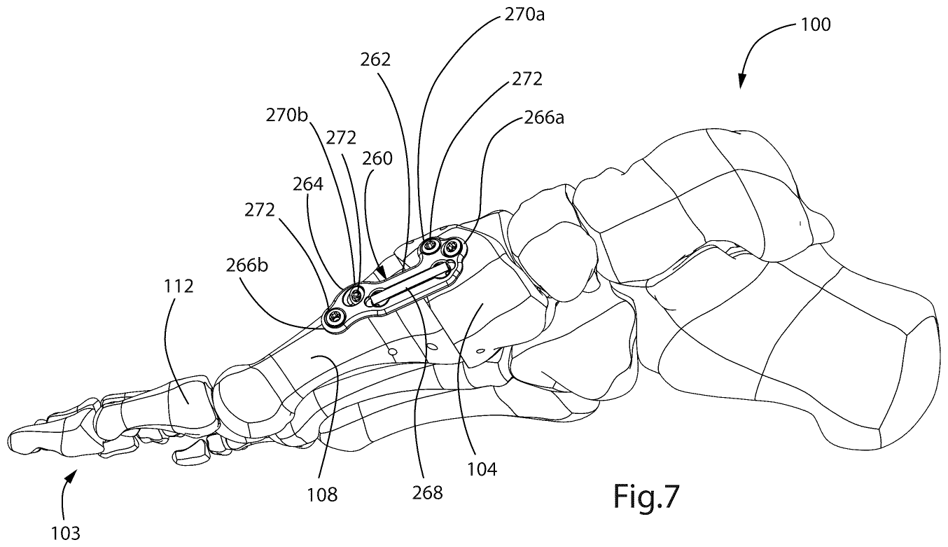

Next, referring to FIG. 7, the first metatarsal 108 can be fixed relative to the medial cuneiform 104 in the corrected configuration 103. In particular, with the bone correction system 150 in the final position and the articulation joint 156 in the locked configuration, a bone plate assembly 260 can attach to each of the medial cuneiform 104 and the first metatarsal 108, thereby securing the medial cuneiform 104 relative to the first metatarsal 108. A bone fixation assembly can include the bone correction system 150, the temporary bone fixation members 168, and the bone plate assembly 260. The bone plate assembly 260 can include an implant such as a bone plate 262 and at least one permanent bone fixation member 272 that are configured to secure the bone plate 262 to the medial cuneiform 104 and the first metatarsal 108.

The bone plate 262 can be contoured to fit against the medial cuneiform 104 and the metatarsal bone 108. The bone plate 262 can be made from titanium, aluminum, steel, other suitable materials known in the orthopedic field. The bone plate 262 can include a plate body 264 having a first end portion 266a and a second end portion 266b opposite the first end portion 266a. The first end portion 266a can be positioned over the medial cuneiform 104, and the second end portion 266b can be positioned over the first metatarsal 108. In some examples, the bone plate assembly 260 can include a compression member, such as a bone staple 268, that is configured to apply a compressive force to the medial cuneiform 104 and the first metatarsal 108. The bone staple 268 can include first and second legs, and a bridge that extends from the first leg to the second leg. The first leg can be driven through the plate body 264 and into the medial cuneiform 104, and the second leg can be driven through the plate body 264 and into the first metatarsal 108. The bone staple 268 can be resilient, and the legs can be deflected when inserted into the respective underlying bone, such that a natural spring force the first and second legs apply the compression force against the medial cuneiform 104 and the first metatarsal 108.

The bone plate assembly 260 can include a plurality of bone fixation apertures that extend through the plate body 264, including at least one first bone fixation aperture 270a that extends through the first end portion 266a and is positioned so as to be aligned with the medial cuneiform 104, and at least one second bone fixation aperture 270b that extends through the second end portion 266b and is positioned so as to be aligned with the first metatarsal 108. In one example, the bone plate 262 can include a pair of first bone fixation apertures 270a and a pair of second bone fixation apertures 270b, though it should be appreciated that the bone plate 262 can include any number of bone fixation apertures 270a-b as desired. Each of the bone fixation apertures 270a-b can be configured to receive a respective permanent bone fixation member 272 to secure the bone plate assembly 260 to the medial cuneiform 104 and the first metatarsal 108. The permanent bone fixation members 272 can be configured as a plurality of fasteners such as threaded bone screws. The threaded bone screws can be driven through the apertures 270a-b and into the medial cuneiform 104 and the first metatarsal 108, respectively. In other examples, the permanent bone fixation members 272 can be configured as pins or other fasteners known in the field of orthopedics. In still other examples, the permanent bone fixation members 272 can be configured as one or more bone staples as described above.

Referring now to FIG. 6D and FIG. 7, it is appreciated that the bone plate 262 fixes the medial cuneiform 104 and the metatarsal bone 108 in the corrected position 103. Accordingly, the once the bone plate 262 has been secured to the medial cuneiform 104 and the metatarsal bone 108, the temporary bone fixation members 168 can be removed from the medial cuneiform 104 and the metatarsal bone 108, and the bone correction system 150 can be removed from the patient's foot 100. Bone ingrowth can occur across the gap to fuse the medial cuneiform 104 to the metatarsal bone 108. The bone plate 262 can remain implanted, or can be removed as desired.

While the first bone fixation member 160 of the bone correction system 150 has been described as configured to be attached to the medial cuneiform 104, and the second bone fixation member has been described as configured to be attached to the first metatarsal 108 in some examples as described herein, it is appreciated in other examples that the first bone fixation member can alternatively be attached to the first metatarsal, and the second bone fixation member can be attached to the medial cuneiform as desired.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

Several illustrative examples of systems and methods have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various examples. All possible combinations and subcombinations of elements and components described herein are intended to be included in this disclosure. No single feature or group of features is necessary or indispensable.

Certain features that are described in this disclosure in the context of separate examples can also be implemented in combination in a single example. Conversely, various features that are described in the context of a single example also can be implemented in multiple examples separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some examples of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some examples. Also, the separation of various components in the examples described above should not be understood as requiring such separation in all examples, and it should be understood that the described components and systems can generally be integrated together in a single product or packaged into multiple products. Additionally, some examples are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures or description herein. For example, various functionalities provided by the illustrated modules can be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification can be included in any example.

In summary, various examples of systems and related methods have been disclosed. This disclosure extends beyond the specifically disclosed examples to other alternative examples and/or other uses of the examples, as well as to certain modifications and equivalents thereof. Moreover, this disclosure expressly contemplates that various features and aspects of the disclosed examples can be combined with, or substituted for, one another. Accordingly, the scope of this disclosure should not be limited by the particular disclosed examples described above, but should be determined only by a fair reading of the claims.

1. A bone correction system comprising:
a first bone attachment member having at least one first temporary bone fixation aperture configured to receive a respective at least one first temporary bone fixation member to attach the first bone attachment member to a medial cuneiform;
a second bone attachment member having at least one second temporary bone fixation aperture configured to receive a respective at least one second temporary bone fixation member to attach the second bone attachment member to a first metatarsal;
a joint connected between the first bone attachment portion and the second bone attachment portion, wherein the joint is configured to articulate the second bone attachment member relative to the first bone attachment member so as to 1) rotate the first bone attachment member about an axis of rotation that extends along a direction that is substantially parallel to a long axis of the first metatarsal, and angulate the first bone attachment member so as to adjust an intermetatarsal angle defined by the first metatarsal and a second metatarsal adjacent the first metatarsal.

2. The bone correction system of claim 1, wherein the joint is configured to articulate the second bone attachment member relative to the first bone attachment member in three orthogonal planes.

3. The bone correction system of claim 2, wherein the second bone attachment member is translatable relative to the first bone attachment member along a shaft.

4. The bone correction system of claim 3, further comprising a translation actuator that is rotatably coupled to the second bone engagement member, translatably fixed to the second bone engagement member, and threadedly mated to the shaft, wherein selective rotation of the translation actuator about the shaft in first and second direction of rotations causes the actuator to drive the second bone engagement member to translate along the shaft toward and away from the first bone engagement member, respectively.

5. The bone correction system of claim 3, wherein the shaft defines an articulation surface of the joint.

6. The bone correction system of claim 5, configured to iterate the joint between a locked configuration and an unlocked configuration.

7. The bone correction system of claim 6, wherein the first bone attachment member comprises a collet that cooperates with a seat to capture the articulation surface between the collet and a seat when the articulation joint is in the locked configuration.

8. The bone correction system of claim 7, comprising an actuator that is configured to drive the collet toward the seat so as to capture the articulation surface between the collet and a seat when the articulation joint is in the locked configuration.

9. The bone correction system of claim 7, wherein the articulation surface is configured to articulate with respect to each of the collet and the seat when the articulation joint is in the unlocked configuration.

10. A method for correcting alignment between a first bone portion and a second bone portion, the method comprising:
attaching a first bone attachment member of a bone correction system to the first bone portion;
attaching a second bone attachment member of the bone correction system to the second bone portion, wherein a joint is connected between the first bone attachment member and the second bone attachment member;
after the attaching steps, applying a force that causes the joint to articulate the second bone attachment member with respect to the first bone attachment member in at least two orthogonal planes, thereby causing the second bone portion to move relative to the first bone portion in the at least two orthogonal planes.

11. The method of claim 10, wherein the two orthogonal planes comprises rotation about an axis of rotation, and an axis of IM angulation.

12. The method of claim 10, wherein the joint further angulates in a pitch direction that is orthogonal to each of the axis of rotation and the axis of IM angulation.

13. The method of claim 10, wherein the joint is a universal joint, and the applying step causes the joint to articulate the second bone attachment member with respect to the first bone attachment member in three orthogonal planes.

14. The method of claim 13, further comprising the step of translating the second bone attachment member with respect to the first bone attachment member.

15. The method of claim 13, wherein the translating step comprises causing the second bone attachment member to translate along a shaft that is connected between the first and second bone attachment members.

16. The method of claim 15, wherein the translating step comprises rotating an actuator relative to the second bone attachment member that is translatably fixed to the actuator, which causes the actuator to threadedly translate the second bone attachment member along the shaft.

17. The method of claim 15, wherein the second bone attachment member is prevented from rotating along a central axis of the shaft.

18. The method of claim 10, further comprising iterating the articulation joint between a locked configuration whereby the articulation joint is prevented from articulating, and an unlocked configuration whereby the articulation joint is configured to articulate in the at least two orthogonal planes.

19. The method of claim 10, wherein of iterating the articulation joint to the locked configuration comprises capturing an articulation surface of the joint between a collet and a seat.

20. The method of claim 10, wherein the first bone portion comprises a medial cuneiform, and the second bone portion comprises a first metatarsal.
